(19) United States
(12) Patent Application Publication
(10) Pub. No.: US 2024/0398802 A1
(43) Pub. Date: Dec. 5, 2024

(54) Topical Compositions and Methods for Hair Growth

(71) Applicant: Noor Brands Company, LLC, High Point, NC (US)

(72) Inventors: Adnan Mjalli, High Point, NC (US); Wiley William Hitchcock, Greensboro, NC (US)

(21) Appl. No.: 18/732,027

(22) Filed: Jun. 3, 2024

Related U.S. Application Data
(60) Provisional application No. 63/470,285, filed on Jun. 1, 2023.

(51) Int. Cl. A61K 31/506; A61K 9/00; A61K 31/4188; A61K 31/573; A61K 31/58; A61K 36/537; A61K 47/10; A61K 47/12; A61K 47/20; A61K 47/36; A61P 17/14

(52) U.S. Cl. CPC A61K 31/506; A61K 9/0014; A61K 31/4188; A61K 31/573; A61K 31/58; A61K 36/537; A61K 47/10; A61K 47/12; A61K 47/20; A61K 47/36; A61P 17/14

(57) ABSTRACT

Disclosed are compositions and methods for the prevention and/or treatment of hair loss. The provided compositions comprise a chitosan gel-based carrier for the transdermal delivery of active hair growth agents. The disclosed compositions allow for the use of multiple active agents, including minoxidil, finasteride, and clobetasol propionate, in combination in a topical formulation that exhibits effective delivery of such agents t for treatment of hair loss.

### RELATED APPLICATIONS

This application claims priority to U.S. provisional patent application No. 63/470,285, filed Jun. 1, 2023. The disclosure of U.S. provisional patent application No. 63/470,285 is incorporated by reference in its entirety herein.

### FIELD

The present disclosure relates to topical compositions and methods for the growth of hair.

### BACKGROUND

Alopecia is the loss or absence of hair in an areas where hair is expected to be present. This condition can be localized or diffuse, temporary or permanent, and affect all sexes and age groups. Hair loss typically develops gradually and about 80% of men show signs of male pattern baldness by age 70 years. Women may also experience hair thinning and hair loss. A variety of factors affect hair loss in both men in women including, genetics, anemia, hormone changes, certain infectious diseases, autoimmune diseases, drug use, diet, and physical and emotional distress.

A variety of compounds have been used to treat hair loss, and there are several hair loss prevention products available on the market. Minoxidil is an established pharmacologic treatment for hair loss. Long-term treatment is necessary and is effective in stopping progression of hair loss, but often only partial regrowth can be achieved. Another hair loss prevention product is finasteride, which is typically administered orally at a dosage of 1 mg/day. There are, however, a number of side effects that have been found to be associated with the administration of finasteride orally that may be reduced using topical administration. Clobetasol propionate ointment is also available on the market and is commonly used to stimulate hair regrowth. Certain studies have shown that hair regrowth may not be maintained long-term following treatment with a clobetasol propionate ointment.

Thus, there is a need to develop therapeutic carrier systems capable of transdermally delivering active agents for hair growth. Disclosed herein are compositions useful for treating baldness or alopecia, and/or promoting hair growth, and/or promoting hair follicle development and/or activation, and/or preventing hair loss on an area of the skin of a subject. The disclosed delivery of active hair growth agents in a chitosan-based gel carrier system can be useful for all the above indications.

### SUMMARY OF THE DISCLOSURE

Topical administration of known hair-growing pharmaceutical agents using a chitosan-based carrier allows for targeted site action while allowing plasma levels of the active ingredients to remain low, and thus, limiting potentially harmful side effects. Thus, in a first aspect, disclosed is a composition formulated for topical application comprising: (i) a carrier composition comprising chitosan and (ii) a therapeutically effective amount of one or more hair regrowth active agents. In some embodiments the carrier gel comprises chitosan powder, a gelling catalyst, and a solvent. In some embodiments, the active ingredient comprises at least one of minoxidil, finasteride, or clobetasol propionate.

In a second aspect, disclosed is a method for preventing and/or treating hair loss in a subject comprising topically administering to the subject any one of the compositions disclosed.

In a third aspect, disclosed is a method for preparing any of the disclosed compositions comprising: (i) preparing a carrier comprising chitosan: (ii) preparing a hair growth active agent blend; and (iii) combining the carrier with the active agent blend.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 depicts a schematic representation of a method for preparing a composition for hair growth in accordance with an embodiment of the disclosure.

FIG. 2 depicts photographic illustration of a beta tester at Day 0, Day 14, and Day 33 of treatment with hair regrowth Formulation #1 (NBC-NH001) (TABLE 1) in accordance with an embodiment of the disclosure. The beta tester applied the formulation 2-3 times/day over the course of treatment.

### DETAILED DESCRIPTION

The presently disclosed subject matter now will be described more fully hereinafter with reference to the accompanying description, in which some, but not all embodiments of the presently disclosed subject matter are shown. The disclosed subject matter can be embodied in many different forms and should not be construed as limited to the embodiments set forth herein: rather, these embodiments are provided so that this disclosure will satisfy applicable legal requirements. Like numbers refer to like elements throughout.

Many modifications and other embodiments of the disclosed subject matter set forth herein will come to mind to one skilled in the art to which the disclosed subject matter pertains having the benefit of the teachings presented in the description. Therefore, it is to be understood that the disclosed subject matter is not to be limited to the specific embodiments disclosed herein and that modifications and other embodiments are intended to be included within the scope of the appended claims. Although specific terms are employed herein, they are used in a generic and descriptive sense only and not for purposes of limitation.

### Terms and Definitions

In order for the disclosure to be more readily understood, certain terms are first defined. Additional definitions for the following terms and other terms are set forth throughout the specification. While the following terms are believed to be well understood by one of ordinary skill in the art, the following definitions are set forth to facilitate explanation of the presently disclosed subject matter. Unless otherwise defined, all technical and scientific terms used herein have the same meaning as commonly understood by one of ordinary skill in the art to which this presently described subject matter belongs. Additionally, any reference referred to as being “incorporated herein” is to be understood as being incorporated in its entirety.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10.

It is further noted that, as used in this specification, the singular forms “a.” “an,” and “the” include plural referents unless expressly and unequivocally limited to one referent. The term “and/or” generally is used to refer to at least one or the other. In some cases, the term “and/or” is used interchangeably with the term “or.” The term “including” is used herein to mean, and is used interchangeably with, the phrase “including but not limited to.” The term “such as” is used herein to mean, and is used interchangeably with, the phrase “such as but not limited to.”

The term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Thus, the term “about” Is used to indicate that a value includes the inherent variation of error for the device, the method being employed to determine the value, or the variation that exists among samples.

The term “buffer solution” is used to indicate a solution that resists a change in pH hen hydrogen ions (H⁺) or hydroxide ions (OH⁻) are added. A buffered solution may be produced by mixing a weak acid with its conjugate base. The buffer solution may be added to water to create “buffered water.”

The term “gelling catalyst” is used to refer to any substance that can increase the rate of a reaction of converting an inorganic colloidal suspension (sol) into a gel (i.e., gelling).

The term “gel” is used to refer to a sol in which the solid particles are meshed such that a rigid or semi-rigid mixture results. The term “sol” is used to refer to a type of colloid in which solid particles are suspended in a liquid.

The term “topical” or topically” is used to refer to administration or application of the composition to a defined area of the body such as a defined area of skin surface.

The term “transdermal” or “transdermally” refers to the penetration and movement of a biologically active agent through the epidermis and dermis, or epidermis, dermis and hypodermis. Transdermal administration can be accomplished by applying, pasting, rolling, attaching, pouring, pressing, rubbing, etc., of a transdermal preparation onto a skin surface.

The term “solvent” is used to refer to any substance, typically a liquid, in which other substances dissolve.

### Compositions

In one aspect, provided herein is a composition formulated for topical application comprising: (i) a carrier comprising chitosan; and (ii) a therapeutically effective amount of one or more hair growth active agents. The carrier may, in certain embodiments, comprise at least one of a chitosan powder, a gelling catalyst, and/or a solvent.

### Carrier

In some embodiments, the carrier for the active ingredients comprises a chitosan. Chitosan has been demonstrated to have several uses including as an anti-cancer agent, a wound healing agent, and an antimicrobial agent. Alsarra (2009) International Journal of Biological Macromolecules 45:6-21. Chitosan-based gels are ideal carriers for topically delivering therapeutic agents due to their low toxicity, biocompatibility, and non-immunogenic properties.

Chitosan is a deacetylated derivative of chitin that is made by treating the chitin found in the shells of shellfish with an alkaline substance. Chitosans are understood to be a family of binary heteropolysaccharides composed of β-1→4 linked 2-acetamido-2-deoxy-β-d-glucopyranose (GlcNAc, the “acetylated”, i.e., the A unit) and 2-amino-2-deoxy-β-d-glucopyranose (GlcNH2, the “deacetylated”, i.e., the D unit) residues, present in different relative proportion and sequence along the chain. Sacco et al. (2018) Gels 4 (3): 67. The degree of deacetylation of chitosan provides the molar percentage of glucosamine monomeric units and varies from 0 (chitin) to 100 (fully deacetylated chitin). The amount of deacetylation affects the ability of chitosan to undergo the transition to a gel as well as the overall stability of the gel. In certain instances, the carrier gel comprises chitosan. In some embodiments, the chitosan gel is made using deacetylated chitosan powder. In some embodiments, the chitosan powder is at least 85%, at least 90% or at least 95% deacetylated. In some embodiments the composition comprises from about 0.5% to 5% by weight of chitosan powder. In some embodiments, the composition comprises from about 2% to 5% by weight of chitosan powder.

In further embodiments, the carrier comprises water or buffered water. In an embodiment, the chitosan is dispersed in water prior to the addition of a gelling catalyst. The addition of water allows the chitosan to crosslink polymer chains upon addition of a gelling catalyst. In some embodiments, the composition comprises from 25-50% water.

Gelling of chitosan powder can be accomplished by chemical or physical means. Thus, in some embodiments the carrier further comprises a gelling catalyst. However, several known methods for gelling chitosan powder are toxic and are not suitable for biomedical uses. In certain embodiments, the gelling catalyst is non-toxic and is suitable for biomedical uses. In some instances, the gelling catalyst is a weak acid, for example, a sugar acid. Sugar acids are monosaccharides with a carboxyl group at one or both ends of its chain. Gelling catalysts suitable for gelling chitosan powder include, but are not limited to, lactic acid, acetic acid, and glycolic acid. In some embodiments, the composition comprises from about 0.5% to 5% by weight of the gelling catalyst. In some embodiments, the composition comprises from about 0.5% to 5% by weight of the gelling catalyst.

In some embodiments, the carrier further comprises one or more solvents. In certain embodiments, the solvent is a non-aqueous solvent. In some instances, the solvent is also a humectant, or a substance with the ability to draw moisture from the surrounding environment. In some instances, the solvent will also function as a gel plasticizing agent, transdermal vehicle, and moisturizing agent. In some embodiments, the solvent is glycerol. In other embodiments, the solvent is propylene glycol, butylene glycol, or sorbitol. In some embodiments, the composition comprises from about 1.0% to 10% by weight of the solvent. In certain embodiments, the composition comprises from about 0.5% to 5% by weight of the solvent.

### Hair Growth Active Agents

In one embodiment, the topical composition comprises a therapeutically effective amount of one or more hair growth active agents. In some embodiments, the topical composition comprises a blend of hair growth active agents. In some embodiments, the composition comprises at least one, two, three, four, five, six, seven, eight, nine, or ten hair growth active agents. In some instances, the hair growth active agent comprises one or more active pharmaceutical ingredients (APIs). APIs are Food and Drug Administration (FDA) regulated chemical and/or drug components. In certain embodiment the active agent is an FDA approved drug for the treatment of hair loss. In some instances, the active agent in a known agent for promoting hair growth, and/or promoting hair follicle development and/or activation, and/or preventing hair loss on an area of the skin of a subject.

In some embodiments, the active agent is minoxidil. In some embodiments, the active agent is a minoxidil-like compound. Minoxidil is a pyrimidine N-oxide that is pyrimidine-2,4-diamine 3-oxide substituted by a piperidin-1-yl group at position 6. Minoxidil functions as a vasodilator and an antihypertensive agent. Minoxidil is commonly administered orally or topically. Minoxidil's hair growth stimulatory effect may be mediated through its vasodilatory activity, thereby increasing cutaneous blood flow, or due to its direct stimulatory effect on hair follicle cells and forcing them from their resting phase into their active growth phase. In some embodiments, the composition comprises less than or equal to about 10%, 7.5%, 5%, 2.5%, or 1% by weight of minoxidil or a minoxidil-like compound.

In some embodiments, the active agent is finasteride. In some embodiments, the active agent is a finasteride-like compound. Finasteride is a synthetic 4-azasteroid compound and functions as a 5-alpha reductase inhibitor. 5-alpha reductase converts testosterone into dihydrotestosterone (DHT), which contributes to hair loss. Thus, finasteride reduces serum DHT levels by disrupting the conversion of testosterone to DHT. Finasteride is commonly delivered orally: however, oral delivery of finasteride can be associated with chills, cold sweats, confusion, dizziness, faintness, and lightheadedness. However, finasteride can be challenging to deliver topically due to its interaction with the active surface of the surfactants in many topical delivery systems. In some embodiments, the composition comprises less than or equal to about 0.5%, 0.4%, 0.3%, 0.2%, 0.1, or 0.05% by weight of finasteride or a finasteride-like compound.

In some embodiments, the active agent is clobetasol propionate. In some embodiments, the active agent is a clobetasol propionate-like compound (e.g., halobetasol priopionate, betamethasone dipropionate, fluo-cinonide, fluocinolone, betamethasone valerate). Clobetasol propionate acts as an inducer of phospholipase A2 inhibitor proteins. Clobetasol propionate is a synthetic fluorinated corticosteroid. In some embodiments, the composition comprises less than or equal to about 0.2%, 0.1, 0.075%, 0.05%, 0.025%, or 0.01% by weight of clobetasol propionate or a clobetasol proprionate-like compound.

In certain embodiments, the composition comprises a blend of two or more active agents. In some instances, the active agent blend comprises two or more of minoxidil, finasteride, and clobetasol propionate or minoxidil-like, finasteride-like, and clobetasol propionate-like compounds. Delivery of two or more active agents together and topically can be challenging due to the size and polarity of the agents. The unique properties of the chitosan-based gel surprisingly allow for delivery of multiple active agents simultaneously.

In certain embodiments, the two or more active agents comprise at least one water soluble agent. In some embodiments, the two or more active agents comprise at least one lipid soluble agent. In certain embodiments, the two or more active agents comprise at least one water soluble agent and at least one lipid soluble agent.

Thus, the disclosed chitosan-based gel carrier/delivery system is advantageous in that it is capable of delivering at least 1, 2, 3, 4, 5, 6, 7, 8, 9, or 10 or more active agents simultaneously. Additionally, the chitosan-based gel carrier/delivery system is capable of delivering agents with differing solubility (e.g., lipid vs water) simultaneously.

In some embodiments the therapeutic agents, including the hair growth active ingredients are in powdered form. Thus, in some embodiments, the hair growth active ingredients are dissolved to form a hair growth active agent blend solution. In some embodiments, the powdered therapeutic agents may be dissolved using a primary non-aqueous solvent. In addition to being capable of dissolving the powdered therapeutic agents, the solvent may have other characteristics. Thus, in some embodiments, the solvent is suitable for biomedical uses, including skin contact, has low toxicity, and serves as a quick-drying agent. In some embodiments, the solvent is a lower alcohol. Lower alcohols are colorless liquids at normal temperatures and include ethanol, propanol, and isopropanol. In some embodiments, the solvent is ethanol. In other embodiments, the solvent is a liniment base. Any liniment base known in the art is suitable for use as a solvent. Examples of such liniment bases include mixtures composed of 10-70 parts by weight of an alcohol such as but not limited to a monohydric alcohol (e.g., ethanol, propanol, isopropyl alcohol), a polyhydric alcohol (e.g., polyethylene glycol, propylene glycol, butylene glycol, or the like), up to 55 parts by weight of water, up to 60 parts by weight of a fatty acid ester (e.g., an ester of adipic acid, sebacic acid, or myristic acid) and up to 10 parts by weight of a surfactant (e.g., polyoxyethylene alkyl ether). In certain embodiments, neutralizing agents (e.g., for pH adjustment), tackifiers (e.g., methyl cellulose, carboxyvinyl polymer, or hydroxypropyl cellulose), rash-preventing agents, and other additives (e.g., salicylic acid, methyl salicylate, glycol salicylate, 1-menthol, camphor: peppermint oil, capsicum, extract, nonylic vanillylamide, crotamiton, Azone, propylene carbonate, or diisopropyl adipate) may also be added in the liniment.

In some embodiments, the powdered therapeutic agents may be dissolved using dimethyl sulfoxide (DMSO), an organic form of sulfur. DMSO is currently used for the treatment of inflammatory conditions and cancer and has been demonstrated to be effective a topical agent. DMSO is able to both penetrate skin and aid dermal penetration of other therapeutics. Brien et al., Osteoarthritis and Cartilage (2008) 16:1277-1288. Thus, in some embodiments, DMSO functions as an analgesic, a solvent, and a facilitator of therapeutic transdermal delivery. In some embodiments, the topical composition comprises about 1-10%, about 2-5%, or about 2.5% DMSO by weight.

In some embodiments, the composition is a topical formulation. In certain embodiments, the topical formulation is a gel, cream, ointment, foam, powder, emulsion, lotion, a spray, or any other topical formulation generally known in the art. In certain embodiments, the topical formulation is a clear gel.

In some embodiments the composition is capable of being stored for at least 6 months, 1 year, 2 years, or 5 years or more at 4-40° C.

### Hair Growth Functional Agents

In some embodiments, the topical composition comprises a therapeutically effective amount of one or more hair growth functional agents. In some embodiments, functional agents are non-FDA regulated agents known to work synergistically with one or more active agents to promote hair growth, and/or promote hair follicle development and/or activation, and/or prevent hair loss on an area of the skin of a subject. For example, functional agents may include nutritional supplements, vitamins, herbals, and/or natural oils with demonstrated ability to promote hair growth and/or prevent hair loss.

In some instances, the active agent further comprises one or more vitamins. In some embodiments, the functional agent is biotin (i.e., vitamin B7 or vitamin H). Biotin is a water-soluble essential B vitamin. B vitamins also help the body metabolize fats and protein and are needed for healthy skin, hair, eyes, and liver. Biotin functions as a cofactor for carboxylase enzymes in several metabolic pathways, including mitochondrial carboxylases in hair roots. In some embodiments, the composition comprises less than or equal to about 0.5%, 0.4%, 0.3%, 0.2%, 0.1, or 0.05% by weight of biotin powder.

In some embodiments, the composition comprises one or more essential oils. In some embodiments, the functional agent is rosemary oil. Rosemary oil is derived from the rosemary plant (Rosmarinus officinalis) whose leaves contain two phenolic diterpenes, carnosic acid and carnosol. These phenolic diterpenes provide protection against oxidative stress. In particular, carnosic acid, a phenolic, lipid-soluble compound with anti-inflammatory and antioxidant properties is understood to increase blood flow. Some studies suggest that rosemary oil may help to stimulate hair growth and slow hair loss. In some embodiments, the composition comprises less than or equal to about 0.5%, 0.4%, 0.3%, 0.2%, 0.1, 0.075%, 0.05%, 0.025%, or 0.01% by weight of rosemary oil.

In certain embodiments, the composition comprises a blend of two or more functional agents. In some instances, the functional agent blend comprises rosemary oil and biotin. Delivery of one or more functional agents together with one or more active agents topically can be challenging due to the size and polarity of the agents. Additionally, the chitosan-based gel carrier/delivery system is capable of delivering agents with differing solubility (e.g., lipid vs water) simultaneously. The unique properties of the chitosan-based gel surprisingly allow for delivery of multiple active agents and functional agents simultaneously.

Thus, the disclosed chitosan-based gel carrier/delivery system is advantageous in that it is capable of delivering at least 1, 2, 3, 4, 5, 6, 7, 8, 9, or 10 or more active agents with at least 1, 2, 3, 4, 5, 6, 7, 8, 9, or 10 or more functional agents simultaneously.

In some embodiments the functional agents are in powdered form. Thus, in some embodiments, the hair growth functional agents are dissolved to form a hair growth functional agent blend solution. In certain embodiments, the functional agents are in liquid form. For example, certain functional agents may be oils. In some embodiments powdered functional agents may be combined with liquid functional agents to for form a functional agent blend.

In some embodiments, the powdered functional agents may be dissolved using a primary non-aqueous solvent. In addition to being capable of dissolving the powdered functional agents, the solvent may have other characteristics. Thus, in some embodiments, the solvent is suitable for biomedical uses, including skin contact, has low toxicity, and serves as a quick-drying agent. In some embodiments, the solvent is a lower alcohol. Lower alcohols are colorless liquids at normal temperatures and include ethanol, propanol, and isopropanol. In some embodiments, the solvent is ethanol. In other embodiments, the solvent is a liniment base. Any liniment base known in the art is suitable for use as a solvent. Examples of such liniment bases include mixtures composed of 10-70 parts by weight of an alcohol such as but not limited to a monohydric alcohol (e.g., ethanol, propanol, isopropyl alcohol), a polyhydric alcohol (e.g., polyethylene glycol, propylene glycol, butylene glycol, or the like), up to 55 parts by weight of water, up to 60 parts by weight of a fatty acid ester (e.g., an ester of adipic acid, sebacic acid, or myristic acid) and up to 10 parts by weight of a surfactant (e.g., polyoxyethylene alkyl ether). In certain embodiments, neutralizing agents (e.g., for pH adjustment), tackifiers (e.g., methyl cellulose, carboxyvinyl polymer, or hydroxypropyl cellulose), rash-preventing agents, and other additives (e.g., salicylic acid, methyl salicylate, glycol salicylate, 1-menthol, camphor: peppermint oil, capsicum, extract, nonylic vanillylamide, crotamiton, Azone®, propylene carbonate, or diisopropyl adipate) may also be added in the liniment.

In some embodiments, the powdered therapeutic agents may be dissolved using dimethyl sulfoxide (DMSO), an organic form of sulfur. DMSO is currently used for the treatment of inflammatory conditions and cancer and has been demonstrated to be effective a topical agent. DMSO is able to both penetrate skin and aid dermal penetration of other therapeutics. Brien et al., Osteoarthritis and Cartilage (2008) 16:1277-1288. Thus, in some embodiments, DMSO functions as an analgesic, a solvent, and a facilitator of therapeutic transdermal delivery. In some embodiments, the topical composition comprises about 1-10%, about 2-5%, or about 2.5% DMSO by weight.

In some embodiments, the composition is a topical formulation. In certain embodiments, the topical formulation is a clear gel. In some embodiments, stable gel formulations are clear.

The stability of the formulation is an important factor in determining the suitability of the formulation for commercial use. In certain embodiments, the compositions described herein provide advantages including stability at 3 months, 6 months, 1 year or more at 4-40° C. as reflected in the lack of changes in viscosity, color, precipitation, crystallization, and phase/layer separation, In certain embodiments, the disclosed gel formulations are able to maintain the active and functional agents without significant degradation over a period of time and over a range of temperatures. In some embodiments the composition is capable of being stored for at least 3 months, 6 months, 1 year, 2 years, or 5 years or more at 4-40° C. without significant degradation. In some embodiments, the rate of degradation is less than 2.0, 1.0, 0.9, 0.8, 0.7, 0.6, 0.5, 0.4, 0.3, 0.2, or less than 0.1%, over the course of 6 or more months at room temperature. In some embodiments, the compositions disclosed herein remain stable following one or more freeze-thaw cycles.

### Methods of Treatment

In other embodiments, provided are methods for preventing and/or treating hair loss. In certain instances, the methods may be used to treat baldness or alopecia, prevent hair loss, promote hair growth, and/or promote hair follicle development and/or activation on an area of the skin of a subject. The subject may be any animal. In an embodiment, the subject is a human. In some embodiments, the subject has experienced hair loss and/or thinning. In certain embodiments, the method comprises topically administering to the subject any one of the described compositions comprising a therapeutically effective amount of one or more active agents and/or functional agents.

In some embodiments, the method for preventing and/or treating hair loss in a subject comprises topically administering to the subject any one of the compositions described herein to the skin (e.g., the scalp). In some embodiments, the composition is applied to a target zone of the scalp (e.g., a bald spot).

Transdermal delivery can improve the efficiency and therapeutic bioavailability, in part, by avoiding the first-pass metabolism that impacts oral drug delivery. Transdermal delivery of therapeutics primarily occurs via stratum corneum, which consists of dead, keratinized epidermal cells. Delivery of drugs across the epidermal cells can be challenging. Thus, in certain embodiments, the size and properties of the therapeutics as well as the properties of the delivery system determine whether a therapeutic can be delivered transdermally.

In some embodiments of the method, the one or more active hair growth agents and/or functional hair growth agents are delivered in a time-release manner. In some embodiments, application of one or more of the compositions described herein results in the formation of a film or bioadhesive on the surface of the skin. The unique chemical and physical properties of the chitosan-based gel carrier may result in the formation of a thin, patch-like film when the gel is applied to skin. The ability of the chitosan-based gel formulation to form a film is significant in that it overcomes several of the disadvantages and limitations of other delivery systems. For example, lotions, ointments, and creams are easily wiped off and require frequent re-application. The use of patches and bandages is also not ideal in that they are prone to causing skin irritation and are difficult to fit over certain areas of the body. The film-forming delivery system described herein allows for the carrier to remain in contact with the skin for prolonged periods of time and deliver the therapeutics in a time-release manner.

Thus, in some embodiments, the carrier allows for increased therapeutic delivery efficiency. Due to the increased efficiency in therapeutic delivery, lower concentrations of therapeutic agents may be used in the compositions and methods disclosed herein. For example, the method may comprise topically administering a composition comprising less than or equal to 5%, 2%, or 1% minoxidil or a minoxidil-like compound to the surface of the skin. In some embodiments, the method comprises topically administering a composition comprising less than or equal to 0.5%, 0.2%, or 0.1% finasteride or a finasteride compound to the surface of the skin. In certain instances, the method comprises topically administering a composition comprising less than or equal to 0.1%, 0.05%, 0.02%, or 0.01% clobetasol propionate or a clobetasol proprionate compound to the surface of the skin.

The ability of the chitosan-based gel formulation to stably carry multiple therapeutic agents in the same formulation provides a significant advantage in that these therapeutics are able to be delivered together to create a synergistic effect. In some embodiments, the method comprises topically administering a composition comprising at least one, two, three, four, five, six, seven, eight, nine, or ten active hair growth agents and/or at least one, two, three, four, five, six, seven, eight, nine, or ten functional hair growth agents. Topical sites treatable through the use of the compositions described herein include, but are not limited to, the scalp.

In some embodiments, the disclosed compositions provide certain advantages including drying time, ability to adhere to skin, spreadability, and greater absorption of active agents and functional agents.

In some embodiments, the composition is applied preemptively, retroactively or both preemptively and retroactively. Thus, in some embodiments, the composition is used proactively to prevent hair loss. In certain embodiments, the composition is used retroactively to promote hair growth and/or prevent further hair loss.

In some embodiments, the methods and compositions described herein may be used for the prevention of hair loss. In other embodiments, the methods and compositions described herein may be used for the treatment of hair loss. In certain instances, the methods and compositions described herein may be used for treatment of hair loss on the scalp.

The compositions can be applied by various physical means, including but not limited to applicator pads, swabs, roller bottles, droppers, or other devices capable of applying the compositions in a thin film. The compositions can be applied with any means known in the art so long as when applied to the area of the skin, the composition will stay in place, i.e., without run-off, for sufficient time, to permit an individual to spread and retain the composition over and on the affected area. In some embodiments, the composition is applied directly to the skin. In some instances, the applied composition is massaged into the skin. For example, following application, the composition may be massaged into the skin using 2-3 fingertips until the treated area is moist but not wet. In some embodiments, the applied composition is allowed to air-dry.

In some embodiments, the topical composition is administered using a patch or bandage. Alternatively and/or additionally, because the chitosan gel-based delivery is able to form a protective skin upon contact with the skin, in some embodiments, it is not necessary to apply the composition to the skin on a separate carrier (e.g., a bandage or dressing). Thus, in some embodiments, the composition is applied directly to the skin.

In certain embodiments, the effect of hair growth active agents, including minoxidil and finasteride is not immediate, but may require continuous use for six months or more in order to achieve noticeable hair growth.

The composition may, in certain embodiments, be provided at least 1, 2, 3, 4, or 5 times a day for at least 1, 2, 3, 4, 5, 6, 7, 8, 9, 10, 11, or 12 days, or for several weeks, or months, or years. Or, longer periods of application may be used.

In some embodiments, the method comprises evaluating the efficacy of treatment using methods known in the art, including but not limited to the unit area trichogram, hair card test, hair weight determination, computed hair analysis, or optical coherence tomography.

### Methods of Preparing Compositions

Also disclosed are methods of preparing compositions formulated to topical application. The methods may be embodied in a variety of ways.

### Methods of Preparing a Composition for Topical Application

In one embodiment, disclosed is a method of preparing a composition formulated for topical application comprising: (i) a carrier comprising chitosan; and (ii) a therapeutically effective amount of one or more hair growth active agents. In some embodiments, the method further comprises preparing a therapeutically effective amount of one or more hair growth functional agents.

In some embodiments, the method comprises preparing a carrier composition comprising chitosan. In some embodiments the method comprises preparing one or more a hair growth active agent blends. In some embodiments, the method further comprises preparing one or more hair growth functional agent blends. Thus, in certain embodiments, disclosed is a method of preparing any of the disclosed compositions comprising: (i) preparing a carrier comprising chitosan: (ii) preparing a hair growth active agent blend; and (iii) combining the carrier with the active agent blend. In some embodiments, the method further comprises combining a functional agent blend with a carrier and/or an active agent blend.

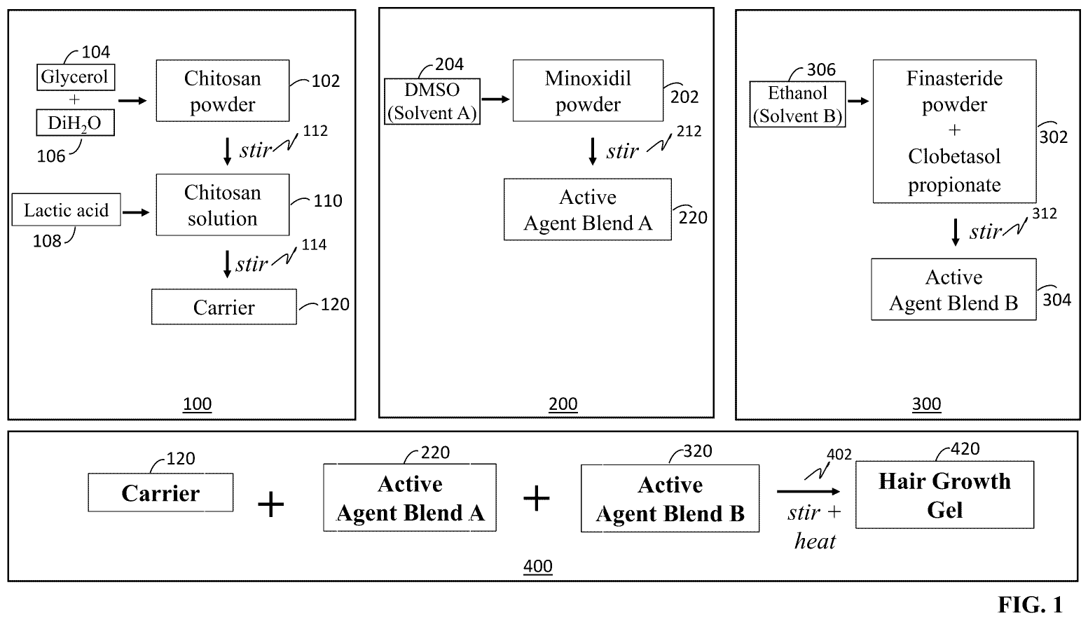

In some embodiments, the method comprises: (i) preparing a carrier comprising chitosan: (ii) preparing an active agent blend and (iii) combining the carrier and the active agent blend. FIG. 1 shows a non-limiting embodiment of the method of preparing a composition for hair growth. As depicted in FIG. 1, a carrier may be prepared (100) by combining chitosan powder, glycerol, diH₂O (deionized H₂O), and lactic acid. Briefly, chitosan powder (102), glycerol (104), and diH₂O (106) are stirred (112) together resulting in a chitosan solution (110) that will disperse, but not dissolve the chitosan powder (102). In some embodiments, lactic acid (108) is slowly added to the dispersed chitosan powder solution so that the chitosan powder is dissolved by the acid and a thick, clear carrier gel forms.

FIG. 1 illustrates a method (200) of preparing a first active agent blend for use in the disclosed compositions. As depicted in FIG. 1, a first active agent blend (Active Agent Blend A) (220) may be prepared (200) by dissolving (212) one or more active agents in a first solvent (Solvent A) (204). As further depicted in FIG. 1, a second active agent blend (Active Agent Blend B) (220) may be prepared (300) by dissolving (312) one or more active agents in a second solvent (Solvent B) (304). For example, in some embodiments, the Solvent A may be DMSO (204). In certain embodiments, powdered forms of the active agent are dissolved in DMSO. For example, minoxidil (202) can be dissolved in DMSO. In some embodiments, powdered forms of the active agents are dissolved in ethanol. For example, finasteride powder and clobetasol propionate (302) can be dissolved (312) in ethanol (306), In some embodiments, only one active agent blend may be used. Or additional active agent blends (e.g. for a total of 3, 4, 5, 6, 7, 8 or 9) active agent blends) may be used.

In some embodiments, the carrier gel can then be combined with the one or more active agent blends (400) to form a hair growth gel composition (420). As shown in FIG. 1, in certain embodiments, the carrier (120) may be first combined with Active Agent Blend A (220) and then combined with the Active Agent Blend B (320), or vice versa. In certain embodiments, Active Agent Blend A (220) and Active Agent Blend B (320) are combined with each other prior to being combined with the carrier (120). In some embodiments, one or more functional agents may be added to either the carrier (100) or Active Agent Blend A (220) or Active Agent Blend B (320) or the carrier/Active Agent Blend solution.

In some embodiments, combining the carrier and active agent blend requires heating (402). In some embodiments, the active agent blend and carrier are mixed together while being heated to at least 40, 45, 50, or 55° C. In certain instances, the carrier and active agent blend are mixed together for at least 1, 2, 3, 4, 5, 6, 7, 8, 9, 10, 11, 12, 13, 14, 15, 16, 17, 18, 19 20, 21, 22, 23, 24, or more hours.

### EXAMPLES

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter.

The following examples describe methods for the preparations and use of a topical hair growth composition.

### Example 1. Preparation of a Hair Growth Gel

### Materials

The following materials were used in the examples and methods below:
1. Chitosan Powder, ≥95% deacetylation
2. Lactic Acid, 85%, CAS #50-21-5
3. Glycerol, 99%+, CAS #56-81-5
4. DiH2O, CAS #7732-18-5
5. Ethanol, 200 proof, CAS #64-17-5
6. Dimethyl Sulfoxide, “DMSO”, 99%, CAS #67-68-5
7. Minoxidil, CAS #38304-91-5, Spectrum Chems #M1138-100GM, 100 g
8. Finasteride, CAS #98319-26-7, Spectrum Chems #F1300-5GM, 5 g
9. Clobetasol Propionate, CAS #25122-46-7, Spectrum Chems #C1785-1GM, 1 g

A chitosan gel was prepared by combining 40.0 g of deacetylated chitosan powder, 100 g of glycerol, and 820 g of deionized water (DiH₂O) into a bottle. The solution was then mixed until the chitosan powder is dispersed. Next, 40.0 g of lactic acid was added to the chitosan solution while stirring until a thick, clear gel of 4.0% chitosan gel (Solution 1) was formed.

Next an active ingredient was prepared. First, 50.0 g of minoxidil powder was combined with 50.0 g of DMSO and gently swirled into a white solution (Solution 2). Second, 1.0 g of finasteride powder, 0.5 g of clobetasol propionate and 498.5 g of ethanol were combined and the contents stirred into solution (Solution 3).

The hair growth active agent blend solution (TABLE 1) was prepared by combining 400 g of 4.0% Chitosan Gel (Solution 1) into 100 g of DMSO/Minoxidil Solution 2 and mixing using a stir bar until the Chitosan Gel (Solution 1) loosely suspended Solution 2. Next, 500 g of Solution 3 was added into the stirring solutions (Solutions 1 and 2) and allowed to blend at about 50° C. until the mixture of the three solutions changed from cloudy to clear. The resulting mixture was a clear, bubbly gel. In some instances, the final mixture (i.e., Solutions 1, 2 and 3) may need to blend for several hours or overnight to avoid clumping.

Using the method described above, various formulations were prepared according to the specifications provided in Tables 1-6.

TABLE 1

| Formulation # 1 | | | |
|---|---|---|---|
| NBC-NH001 | Materials: | g/L | % |
| Carrier: | Chitosan Gel | 400.0 | 40.0 |
| | DMSO | 50.0 | 5.0 |
| | Ethanol | 498.5 | 49.85 |
| Hair Growth | Minoxidil | 50.0 | 5.0 |
| Blend Active | Finasteride | 1.0 | 0.1 |
| Ingredients | Clobetasol | 0.5 | 0.05 |
| | propionate | | |

TABLE 2

| Formulation # 2 | | | |
|---|---|---|---|
| NBC-NH002 | Materials: | g/L | % |
| Carrier: | Chitosan Gel | 400.0 | 40.0 |
| | DMSO | 50.0 | 5.0 |
| | Ethanol | 498.5 | 50.0 |
| Hair Growth | Minoxidil | 50.0 | 5.0 |
| Blend Active | | | |
| Ingredients | | | |

TABLE 3

| Formulation # 3 | | | |
|---|---|---|---|
| NBC-NH003 | Materials: | g/L | % |
| Carrier: | Chitosan Gel | 400.0 | 40.0 |
| | DMSO | 50.0 | 5.0 |
| | Ethanol | 498.5 | 49.9 |
| Hair Growth | Minoxidil | 50.0 | 5.0 |
| Blend Active | Finasteride | 1.0 | 0.1 |
| Ingredients | | | |

TABLE 4

| Formulation # 4 | | | |
|---|---|---|---|
| NBC-NH004 | Materials: | g/L | % |
| Carrier: | Chitosan Gel | 400.0 | 40.0 |
| | DMSO | 50.0 | 5.0 |
| | Ethanol | 498.5 | 49.95 |
| Hair Growth | Minoxidil | 50.0 | 5.0 |
| Blend Active | Clobetasol | 0.5 | 0.05 |
| Ingredients | propionate | | |

TABLE 5

| Formulation # 5 | | | |
|---|---|---|---|
| NBC-NH005 | Materials: | g/L | % |
| Carrier: | Chitosan Gel | 400.0 | 40.0 |
| | DMSO | 50.0 | 5.0 |
| | Ethanol | 498.5 | 49.55 |
| Hair Growth | Minoxidil | 50.0 | 5.0 |
| Blend Active | Clobetasol | 0.5 | 0.05 |
| Ingredients | propionate | | |
| | Finasteride | 1.0 | 0.1 |
| | Biotin powder | 2.0 | 0.2 |
| | Rosemary oil, neat | 1.0 | 0.1 |

TABLE 6

| Formulation # 6 | | | |
|---|---|---|---|
| NBC-NH006 | Materials: | g/L | % |
| Carrier: | Chitosan Gel | 400.0 | 40.0 |
| | DMSO | 50.0 | 5.0 |
| | Ethanol | 498.5 | 49.7 |
| Hair Growth | Minoxidil | 50.0 | 5.0 |
| Blend Active | Biotin powder | 2.0 | 0.2 |
| Ingredients | Rosemary oil, neat | 1.0 | 0.1 |

### Example 2. Stability Testing

Formulation stability was assessed under various conditions. Stable gel formulations are clear and transparent without clouding due to precipitation of ingredients, crystal formation over time, color changes, and/or separation layers. Thus, formulations were observed during testing for any changes in appearance and/or consistency. All stability tests were performed in a closed container and preferably done in an end-use bottle/container to mimic an end-user experience. Formulation stability was assessed in both light and dark environments to evaluate any light (normally UV) sensitivity (data not shown). Any light sensitivity can be overcome using packaging materials generally known in the art for light protection. Testing of formulation stability was also assessed at various temperatures: cool (˜4° C.) (TABLE 7), room temperature (˜20-24° C.) (TABLE 8), and warm (˜55° C.) (TABLE 9). All formulations remained stable across temperatures of 4-55° C. as determined by the lack of clouding due to precipitation of ingredients, crystal formation over time, color changes, and separation layers.

Additionally, formulation stability was assessed across one or more freeze-thaw cycles to evaluate any solubility issues following a freeze-thaw cycle (TABLE 10). Following a freeze-thaw cycle, formulations were observed for any solubility issues. All of the formulations were stable (i.e., remained clear and transparent without clouding due to precipitation of ingredients, crystal formation over time, color changes, and/or separation layers) (TABLE 10). Additionally, all formulations were able to blend back together after thawing with minimal effort (e.g., gentle mixing) (TABLE 10).

TABLE 7
Formulation Stability at 4° C.

| | | Crystal | Color | Separation |
|---|---|---|---|---|
| | Clouding | Formation | Change | Layers |
| Formulation | (Yes/No) | (Yes/No) | (Yes/No) | (Yes/No) |
| NBC-NH001 | No | No | No | No |
| NBC-NH002 | No | No | No | No |
| NBC-NH003 | No | No | No | No |
| NBC-NH004 | No | No | No | No |
| NBC-NH005 | No | No | No | No |
| NBC-NH006 | No | No | No | No |

TABLE 8
Formulation Stability at 20-24° C.

| | | Crystal | Color | Separation |
|---|---|---|---|---|
| | Clouding | Formation | Change | Layers |
| Formulation | (Yes/No) | (Yes/No) | (Yes/No) | (Yes/No) |
| NBC-NH001 | No | No | No | No |
| NBC-NH002 | No | No | No | No |
| NBC-NH003 | No | No | No | No |
| NBC-NH004 | No | No | No | No |
| NBC-NH005 | No | No | No | No |
| NBC-NH006 | No | No | No | No |

TABLE 9
Formulation Stability at 55° C.

| | | Crystal | Color | Separation |
|---|---|---|---|---|
| | Clouding | Formation | Change | Layers |
| Formulation | (Yes/No) | (Yes/No) | (Yes/No) | (Yes/No) |
| NBC-NH001 | No | No | No | No |
| NBC-NH002 | No | No | No | No |
| NBC-NH003 | No | No | No | No |
| NBC-NH004 | No | No | No | No |
| NBC-NH005 | No | No | No | No |
| NBC-NH006 | No | No | No | No |

TABLE 10
Freeze/Thaw: −20° C. to Room Temperature (~22° C.)

| | Freeze/thaw |
|---|---|
| Formulation | stability |
| NBC-NH001 | Pass |
| NBC-NH002 | Pass |
| NBC-NH003 | Pass |
| NBC-NH004 | Pass |
| NBC-NH005 | Pass |
| NBC-NH006 | Pass |

### Example 3. Application and Delivery Efficiency

Application and delivery efficiency were tested by evaluating skin applications. Each formulation was evaluated post-application to the skin of a subject by examining the application area for a period of time following application.

Unsuccessful delivery results in residue being deposited on the skin instead of being transdermally delivered into the skin tissue. The presence of any undelivered material indicates that the formulation was not able to transdermally deliver one or more active and/or functional agents. A successful product application was characterized by a thin-film layer of the chitosan gel that acts like a second skin without any residue. A successful product application may initially feel tacky but will dry and be visually clear within a few (i.e., 5 mins). Some active agents and/or functional agents may alter the speed at which the application site dries or may show a persistent tackiness over time. All formulations disclosed in TABLES 1-6 were found to successfully deliver the active and functional agents without residue being deposited on the skin.

### Example 4. Treatment of Beta Testers with Topical Hair Loss Gel

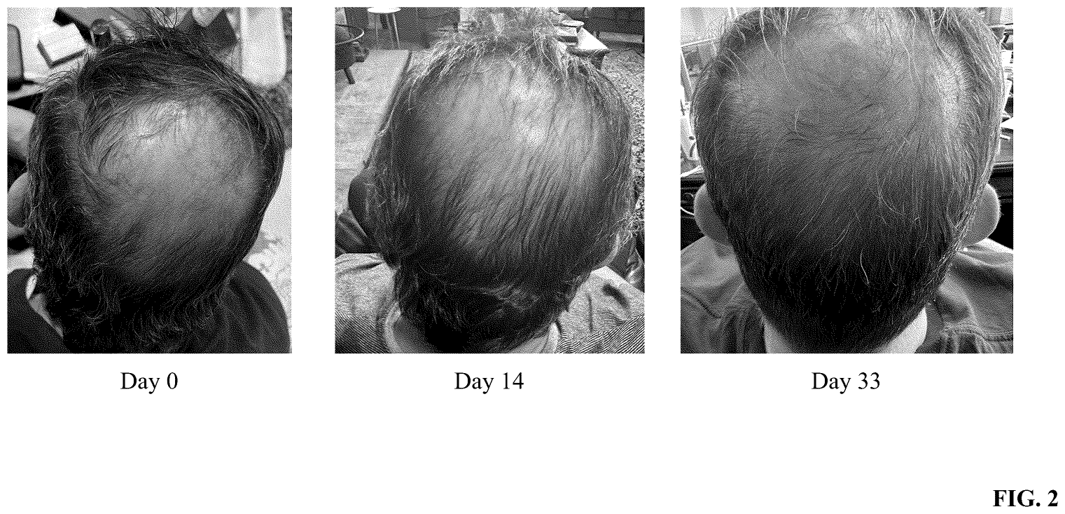

Briefly, beta testers experiencing male pattern baldness were instructed to apply Formulation #1 (NBC-NH001) to bald spots on their scalps using a roller bottle or dropper containing the formulation. Beta testers using the roller bottle were instructed to remove the cap from the bottle and roll the applicator directly against the scalp target zone the directed number of times to cover the area. Beta testers using a dropper bottle were instructed to draw the directed amount of the agent into the dropper and apply to the target zone on the scalp. Following application using the roller bottle or dropper, beta testers were instructed to massage the applied product into the target area with 2-3 fingertips until the area was moist but not wet. The applied product was then allowed to air-dry. Beta testers applied the formulation 2-3 times daily. Approximately 0.1-0.2 g (100-200 μL) of formulation were applied to the treated area per application. Beta testers indicated increased hair growth and fullness by day 33 of treatment as depicted in FIG. 2. Hair growth may be evaluated employing methods known in the art, including but not limited to the unit area trichogram, hair card test, hair weight determination, computed hair analysis, or optical coherence tomography.

### Example 5. Embodiments of Suitable Compositions and Methods

This disclosure includes, but is not limited to, the following embodiments.

A.1 A composition formulated for topical application comprising: (i) a carrier composition comprising chitosan; and (ii) a therapeutically effective amount of one or more hair growth active agents.

A.2 The composition of embodiment A.1, wherein the carrier further comprises a gelling catalyst and a solvent.

A.3 The composition of embodiments A.1-A.2, wherein the composition comprises from about 2% to 5% by weight of chitosan.

A.4 The composition of embodiments A. 1-A.3, wherein the composition comprises from about 0.5% to 5% by weight of the gelling catalyst.

A.5 The composition of embodiments A.1-A.4, wherein the composition comprises from about 0.5% to 5% by weight of the solvent.

A.6 The composition of embodiments A.1-A.5, wherein the gelling catalyst comprises lactic acid.

A.7 The composition of embodiments A.1-A.6, wherein the solvent comprises glycerol.

A.8 The composition of embodiments A.1-A.7, wherein the active agent comprises at least one of minoxidil, finasteride, or clobetasol propionate.

A.9 The composition of embodiments A.1-A.8, further comprising one or more functional agents.

A.10 The composition of embodiments A.1-A.9, wherein the functional agent comprises at least one of biotin or rosemary oil.

A.11 The composition of embodiments A.1-A.10, wherein the composition comprises from about 0.05% to 1.5% by weight of the solvent.

A.12 The composition of embodiments A.1-A.11, wherein the composition comprises less than or equal to about 5% by weight of minoxidil, less than or equal to about 0.1% by weight of finasteride, and less than or equal to about 0.05% by weight of clobetasol propionate.

A.13 The composition of embodiments A.1-A.12, wherein the composition further comprises DMSO

A.14 The composition of embodiments A.1-A.13, The composition of claim 1, wherein the composition further comprises ethanol.

A.15 The composition of embodiments A.1-A.14, wherein the solvent further comprises water or buffered water.

B.1 A method for preventing and/or treating hair loss in a subject comprising topically administering to the subject any one of the compositions of A.1-A.15.

B.2 The method of embodiment B.1, wherein the composition is applied to the skin or scalp.

C.1 The method of preparing any of the compositions of claims A.1-A.15 comprising: (i) preparing a carrier comprising chitosan; (ii) preparing a hair growth active agent blend; and (iii) combining the carrier with the active agent blend.

C.2. The method of embodiment C.1, further comprising preparing a hair growth functional agent blend and combining the functional agent blend with the carrier and active agent blend.

What is claimed:

1. A composition formulated for topical application comprising:
(i) a carrier composition comprising chitosan; and
(ii) a therapeutically effective amount of one or more hair growth active agents.

2. The composition of claim 1, wherein the carrier further comprises a gelling catalyst and a solvent.

3. The composition of claim 1, wherein the composition comprises from about 2% to 5% by weight of chitosan.

4. The composition of claim 1, wherein the composition comprises from about 0.5% to 5% by weight of the gelling catalyst.

5. The composition of claim 1, wherein the composition comprises from about 0.5% to 5% by weight of the solvent.

6. The composition of claim 2, wherein the gelling catalyst comprises lactic acid.

7. The composition of claim 2, wherein the solvent comprises glycerol.

8. The composition of claim 1, wherein the active agent comprises at least one of minoxidil, finasteride, or clobetasol propionate.

9. The composition of claim 1, further comprising one or more functional agents.

10. The composition of claim 9, wherein the functional agent comprises at least one of biotin or rosemary oil.

11. The composition of claim 2, wherein the composition comprises from about 0.05% to 1.5% by weight of the solvent.

12. The composition of claim 8, wherein the composition comprises less than or equal to about 5% by weight of minoxidil, less than or equal to about 0.1% by weight of finasteride, and less than or equal to about 0.05% by weight of clobetasol propionate.

13. The composition of claim 1, wherein the composition further comprises DMSO.

14. The composition of claim 1, wherein the composition further comprises ethanol.

15. The composition of claim 1, wherein the solvent further comprises water or buffered water.

16. A method for preventing and/or treating hair loss in a subject comprising topically administering to the subject the composition of claim 1.

17. The method of claim 16, wherein the composition is applied to the skin or scalp.

18. A method of preparing the composition of claim 1 comprising:
(i) preparing a carrier comprising chitosan;
(ii) preparing a hair growth active agent blend; and
(iii) combining the carrier with the active agent blend.

19. The method of claim 18, further comprising preparing a hair growth functional agent blend and combining the functional agent blend with the carrier and active agent blend.
